(19) United States
(12) Patent Application Publication
(10) Pub. No.: US 2021/0130446 A1
(43) Pub. Date: May 6, 2021

(54) COMPOSITIONS AND METHODS FOR TREATING LIVER DISEASE

(71) Applicant: Children's Hospital Medical Center, Cincinnati, OH (US)

(72) Inventor: Pranav-Kumar Shivakumar, Cincinnati, OH (US)

(21) Appl. No.: 17/043,763

(22) PCT Filed: Apr. 12, 2019
(86) PCT No.: PCT/US19/27177

§ 371 Date: Sep. 30, 2020

Related U.S. Application Data
(60) Provisional application No. 62/656,426, filed on Apr. 12, 2018.

(51) Int. Cl. C07K 16/18

(52) U.S. Cl. CPC C07K 16/18

(57) ABSTRACT

Disclosed are methods of treating a subject, particularly a human individual, more particularly a pediatric individual, having a biliary disorder, via administration of a therapeutically effective amount of a C5 inhibitor. The biliary disorder may include biliary atresia and post-Kasai biliary atresia.

### CROSS-REFERENCE TO RELATED APPLICATIONS

This application claims priority to and benefit of U.S. Provisional Patent Application 62/656,426, filed Apr. 12, 2018, entitled “Targeting Complement C5 as a Novel Approach to Treat Neonatal Biliary Atresia,” the contents of which are incorporated in its entirety for all purposes.

### BACKGROUND

Pediatric cholestatic liver diseases affect a small percentage of children, but therapy results in significant healthcare costs each year. Currently, many of the pediatric cholestatic liver diseases require invasive and costly treatments such as liver transplantation and surgery. An effective and less invasive treatment that is suitable for the pediatric population is not available.

Biliary Atresia (BA), in particular, is a pediatric liver disease restricted to newborn infants with no known medical treatment. BA is a rare neonatal disease manifesting only in the first few weeks of life characterized by ascending obstruction of the biliary tree resulting in severe cholestasis and rapidly progressing biliary cirrhosis. The common histopathological picture is one of inflammatory damage to the intra- and extrahepatic bile ducts with sclerosis and narrowing or obliteration of the biliary tree. BA is a rapidly progressing obliterative disease of the extra- and intra-hepatic bile ducts and represents an extreme spectrum of neonatal cholestasis. Children who develop BA are born jaundice-free; however, within the first weeks of life, the extrahepatic biliary tree develops inflammation leading to duct obstruction and loss of bile flow.

Untreated, this condition leads to cirrhosis and death within the first years of life. BA remains the most common indication for pediatric liver transplantation worldwide. The incidence of B.A is approximately 1:10-15,000 of live births and is classified as a rare disease by NORD (National Organization of Rare Disorders) end NODK, Children who develop BA are born jaundice-free; however, within the first weeks of life, the extrahepatic biliary tree develops inflammation leading to duct obstruction and loss of bile flow. The baby suffers from acholic (chalk-colored) stools, yellowing of skin, enlarged liver and spleen, ascites develops with rapidly progressing liver injury and cirrhosis, and the baby suffers from loss of weight, becomes irritable and has worsening jaundice. Infants with BA are severely ill and may face developmental challenges even after liver transplantation. Infants affected by BA represent an extreme spectrum of neonatal cholestasis and show progressive jaundice and growth retardation. Because of the severe clinical manifestations and limited therapeutic options, most infants progress to end-stage liver cirrhosis, portal hypertension and liver failure eventually needing liver transplantation.

Intraoperative cholangiogram is the only mechanism available for a definitive diagnosis of BA. Because of the progressive nature of the disease, infants, at the time of diagnosis present with a scarred extrahepatic bile duct with varying degrees of intrahepatic inflammation and fibrosis. Surgical intervention by Kasai portoenterostomy (KPE) is the only treatment option, which removes the entire fibrosed biliary tree and surgically recreates an intestinal anastomosis to establish bile flow. While the postsurgical medical management combines nutrition, antibiotics, choleretics, and possibly anti-inflammatory medications, the impact of these practices on the clinical outcome is unclear, and there are no medical therapies available to prevent or reduce the likelihood of ongoing liver injury following a Kasai procedure. Further, infants having a “failed Kasai” will require a liver transplant in infancy to survive, and infants diagnosed too late have too much liver damage to benefit from Kasai and will require early transplant. These two groups of patients encompass about ⅓ to more than half of the BA population. In fact, the Kasai procedure only restores bile flow and 80% of patients still progress to failure.

Post-operative complications are also significant in that some patients, even after successful bile drainage, can still experience cholangitis and succumb to infection. Despite the clinical success of resolving extrahepatic bile duct manifestations of the disease, progression of the liver disease involving intrahepatic bile ducts continue in a majority of children resulting in cirrhosis, with only 13-50% of patients alive with native liver by 2 years of age. In infants progressing to end-stage cirrhosis, liver transplantation is the only option—assuming an average cost of $200-300K per transplant, the economic burden to treat children with BA is approximately —$134 million annually placing significant strain on the affected families as well as on health-care resources and service utilization costs. This is further compounded by a complete lack of medical interventions.

The current nontransplant treatment strategies are at best palliative and primarily make use of steroids in the immediate post-operative period due to their anti-inflammatory properties. However, the role of corticosteroids in improving bile flow is controversial. Indeed, several clinical trials including the most recent and extensive trial of corticosteroid therapy in the US following Kasai (ChiLDREN; START trial: NCT00294684) showed that steroids alone do not prevent the need for liver transplantation. Outcomes from most of these trials strongly suggest the existence of inflammatory footprints beyond the immune-suppressive capacity of, or pathways regulated by steroids. Of significance, in the current pediatric end-stage liver disease (PELD) system, children with BA face the risk of not receiving a liver in a safe and timely manner.

Thus, identification of temporo-spatial effectors of hepatobiliary injury is of paramount significance towards designing novel treatment strategies. The instant invention seeks to address one or more of the aforementioned needs in the art.

### BRIEF SUMMARY

Disclosed are methods of treating a subject, particularly a human individual, more particularly a pediatric individual, having a biliary disorder, via administration of a therapeutically effective amount of a C5 inhibitor. The biliary disorder may include biliary atresia and post-Kasai biliary atresia.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. Complement protein expressions in human livers. Microarray analysis of livers from control subjects (left) and patients with Biliary Atresia (BA) (right) shows decreased expressions of complement component C5 indicating active utilization and consumption. ***P≤0.0001.

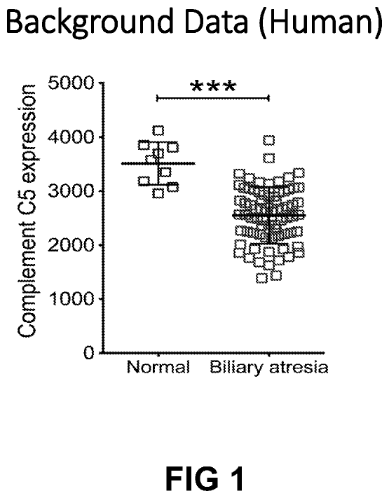

FIG. 2. Complement protein levels in human subjects. High-throughput proteomic analysis using Somalogic SOMAScan platform identified baseline levels of complement C5, C5a and C5b-C6 complex in normal human subjects and elevated expressions in serum from patients with Biliary atresia indicating systemically dysregulated activation of the complement system. **P≤0.001 and *P≤0.05.

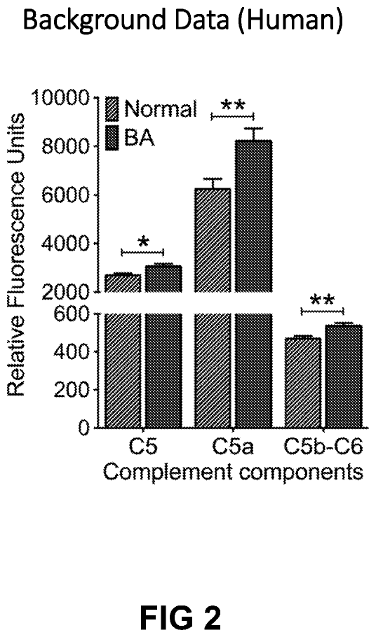

FIG. 3. Experimental neonatal mouse model of Biliary atresia. Newborn mice infected with 1.0×10⁶ pfu (plaque-forming units) of rhesus rotavirus (RRV; non-pathogen to humans) within 24 hours of birth (A) recapitulates symptoms of human BA with severe inflammation of the extrahepatic bile ducts by day 7 after RRV infection (B), progressing to atresia, loss of duct lumen and bile stasis (C) by day 12-14 of life.

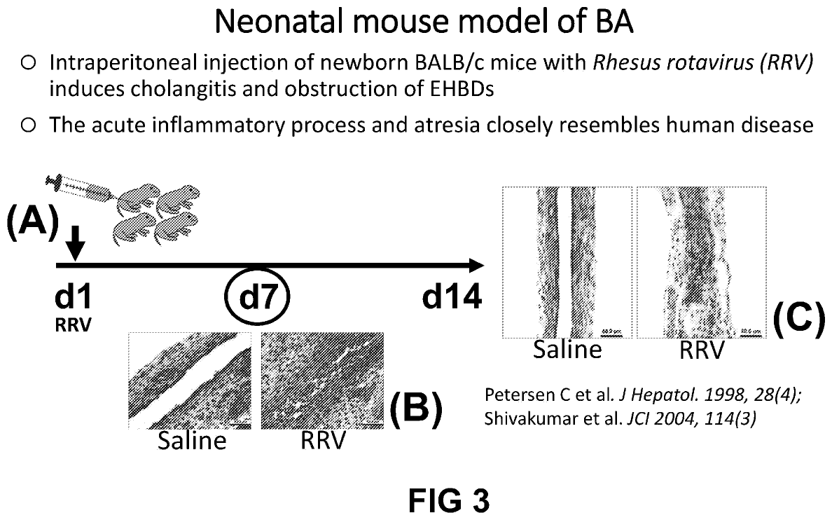

FIG. 4. Plasma concentrations of activated complement C5a. Newborn mice were either injected with 0.9% normal saline (control cohort) or infected with 1.0×10⁶ pfu of RRV within 24 hours of birth and plasma was collected at days 3, 5, 7 and 14 by cardiac puncture. Mouse C5a ELISA was used to quantify plasma levels of C5a. Results show that C5a is highly elevated at days 5 and 7 corresponding to severe bile duct inflammation and obstruction to bile flow. Plasma was pooled from 4-6 mice/group/time-point with N=3-4 specimens/time-point. *P≤0.05, **P≤0.001 and ***P≤0.0001.

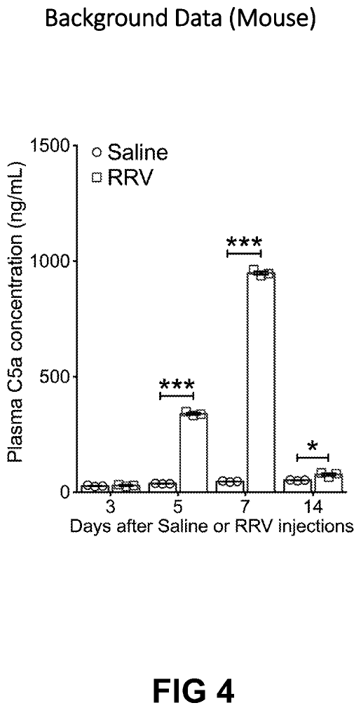

FIG. 5. Blocking Complement C5 prevents growth failure and promotes survival. Newborn mice were either injected with 0.9% normal saline (control cohort) or infected with 1.0×10⁶ pfu of RRV within 24 hours of birth. 10 g of anti-C5 (BB5.1) antibodies were administered daily by subcutaneous injection until day 12 of life and weights were recorded daily. Isotype or untreated RRV-infected mice (red) showed poor growth profiles and failure to thrive (A) and 100% mortality (B) by days 13-14 after RRV infection while treatment with BB5.1 (Anti-mouse C5 mAb (Wang et al., Proc. Natl. Acad. Sci. U.S.A (1996) 93:8563-8568), see also U.S. Pat. No. 10,125,191) prevented growth failure (t) and promoted long-term survival shown by Kaplan-Meier Survival analysis. Weight-gain of saline-injected mice is shown in blue. ***P≤0.0001: RRV-isotype vs BB5.1-treated mice.

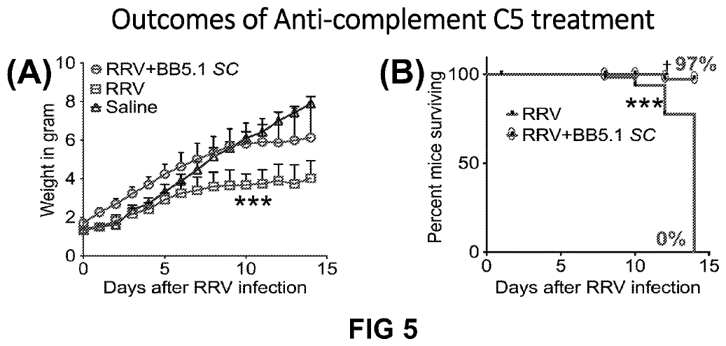

FIG. 6A. Anti-complement C5 treatment prevents EHBD obstruction and atresia. EHBDs were obtained from isotype or anti-complement C5 (BB5.1) treated neonatal/infantile mice infected with 1.0×10⁶ pfu of RRV within 24 hours of birth. Newborn mice received daily doses of 10 μg anti-C5 (BB5.1) antibodies subcutaneously until day 12 of life. Microdissected EHBDs were sectioned at 5 μm thickness, subjected to Hematoxylin and Eosin (H&E) staining and analyzed by light microscopy. BB5.1 treatment prevented the classical EHBD atresia (B, C) phenotype associated with BA (A) and allowed for efficient bile flow. N=10-15 EHBDs/group.

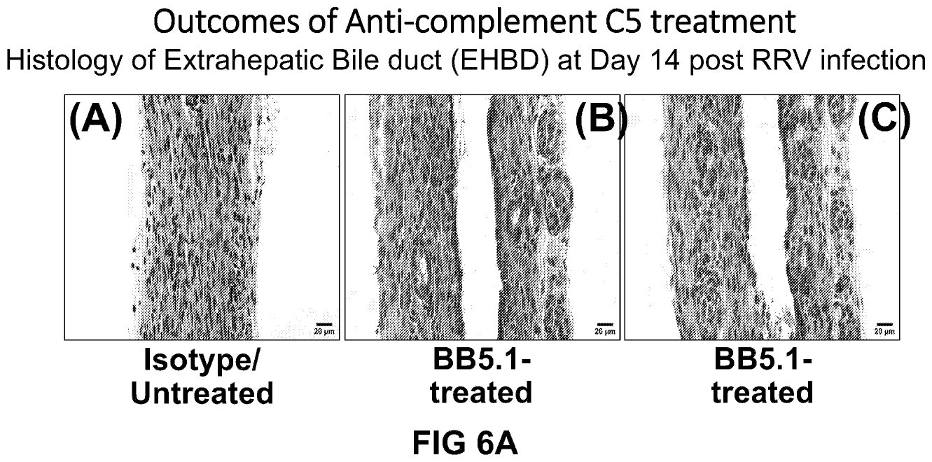

FIG. 6B. Anti-complement C5 treatment prevents EHBD obstruction and atresia. EHBDs were obtained from isotype or anti-complement C5 (BB5.1) treated neonatal/infantile mice infected with 1.0×10⁶ pfu of RRV within 24 hours of birth. Newborn mice received daily doses of 10 μg anti-C5 (BB5.1) antibodies subcutaneously until day 12 of life. Microdissected EHBDs were sectioned at 5 μm thickness, subjected to Hematoxylin and Eosin (H&E) staining and analyzed by light microscopy. BB5.1 treatment prevented the classical EHBD atresia (B, C) phenotype associated with BA (A) and allowed for efficient bile flow. N=10-15 EHBDs/group.

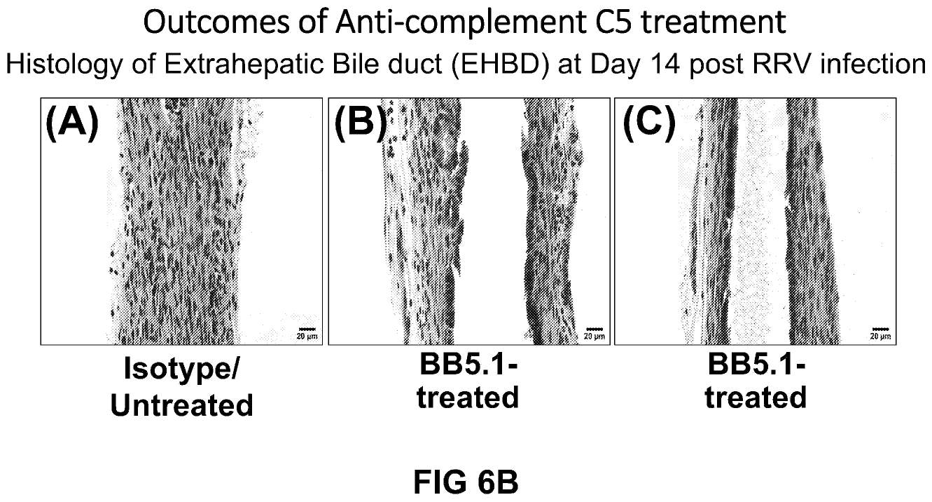

FIG. 7. Blocking Complement C5a prevents growth failure and promotes survival. Newborn mice were either injected with 0.9% normal saline (control cohort) or infected with 1.0×10⁶ pfu of RRV within 24 hours of birth. 13.5 g of anti-C5a (CLS026) antibodies were administered daily by intraperitoneal injection until day 12 of life and weights were recorded daily. Isotype or untreated RRV-infected mice showed poor growth profiles and failure to thrive (A) and 100% mortality (B) by days 13-14 after RRV infection while treatment with CLS026 prevented growth failure (t) and promoted long-term survival shown by Kaplan-Meier Survival analysis. Weight-gain of saline-injected mice is shown with triangles. ***P≤0.0001: RRV-isotype vs CLS026-treated mice. CLS026 is described in, for example Connecting the innate and adaptive immune responses in mouse choroidal neovascularization via the anaphylatoxin C5a and γδT-cells, Beth Coughlin et al., Scientific Reports volume 6, Article number: 23794 (2016).

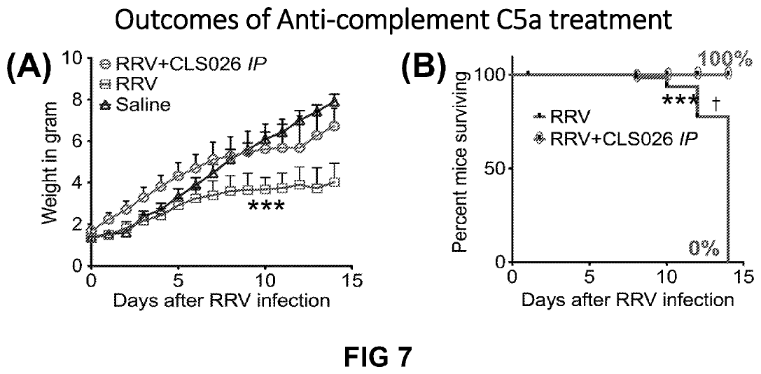

FIG. 8A. Anti-complement C5a treatment prevents extrahepatic bile duct (EHBD) obstruction and atresia. EHBDs were obtained from isotype or anti-complement C5a (CLS026) treated neonatal/infantile mice infected with 1.0×10⁶ pfu of RRV within 24 hours of birth. Newborn mice received daily doses of 13.5 μg anti-C5a (CLS026) antibodies intraperitoneally until day 12 of life. Microdissected EHBDs were sectioned at 5 μm thickness, subjected to Hematoxylin and Eosin (H&E) staining and analyzed by light microscopy. CLSO26 treatment prevented classical EHBD atresia (B, C) phenotype associated with BA (A) and allowed for efficient bile flow. N=10-15 EHBDs/group.

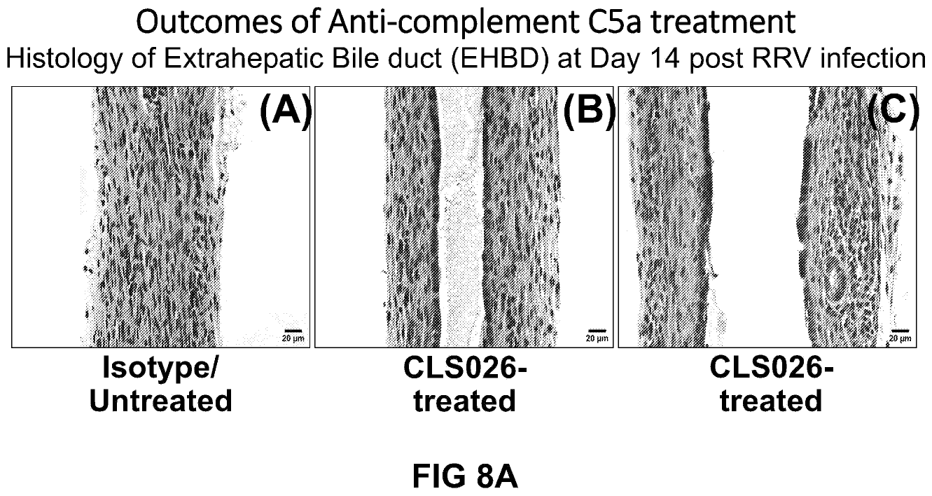

FIG. 8B. Anti-complement C5a treatment prevents EHBD obstruction and atresia. EHBDs were obtained from isotype or anti-complement C5a (CLSO26) treated neonatal/infantile mice infected with 1.0×10⁶ pfu of RRV within 24 hours of birth. Newborn mice received daily doses of 13.5 μg anti-C5a (CLSO26) antibodies intraperitoneally until day 12 of life. Microdissected EHBDs were sectioned at 5 μm thickness, subjected to Hematoxylin and Eosin (H&E) staining and analyzed by light microscopy. CLSO26 treatment prevented classical EHBD atresia (B, C) phenotype associated with BA (A) and allowed for efficient bile flow. N=10-15 EHBDs/group.

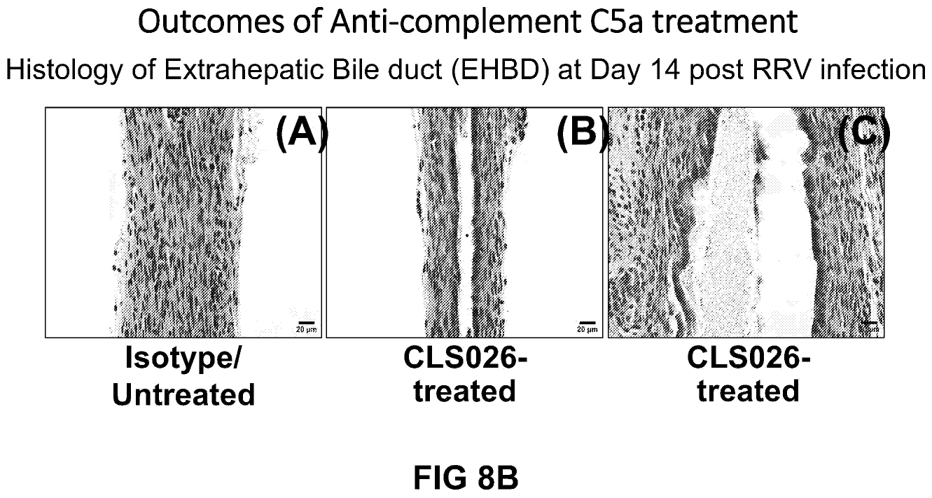

FIG. 9. Development of a novel infantile mouse model of liver fibrosis. Neonatal mice were either injected with 0.9% normal saline (control mice) or infected with RRV on day 3 of life. Livers harvested at day 19 from control mice and stained with Sirius Red showed normal liver architecture (A), while livers from RRV-infected mice show prominent development of hepatic biliary fibrosis (B) with portal-portal bridging (C) and moderate to severe portal inflammation. Development of this novel mouse model allowed for critical testing of anti-complement C5 and C5a to block or attenuate liver fibrosis—the primary indication for liver transplantation in children with BA. Mag.: 100×.

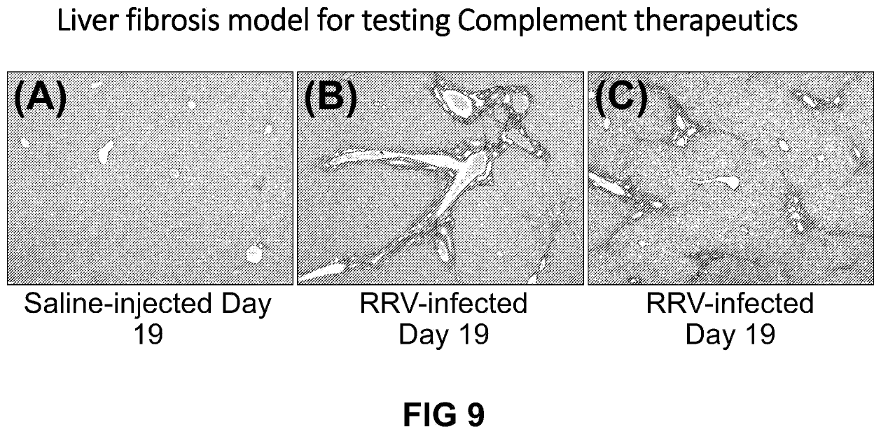

FIG. 10. Blocking Complement C5 promotes survival in mice with liver fibrosis. Neonatal mice were infected with RRV on day 3 of life and treated subcutaneously with isotype antibodies daily or with 10 g of anti-C5 (BB5.1) antibodies until day 19 of life. (A) While BB5.1 treatments did not significantly affect weight gain or growth profiles (squares vs circles), anti-complement therapy significantly suppressed mortality and promoted long-term survival (t) assessed by Kaplan-Meier analysis; isotype-treated or untreated mice showed ˜29% mortality by day 19 after infection. **P≤0.001: RRV-isotype/untreated vs BB5.1-treated mice.

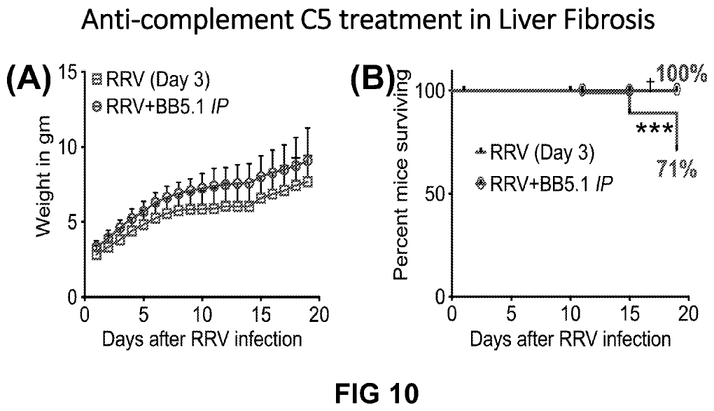

FIG. 11. Anti-complement C5 treatment dampens portal inflammation. Neonatal mice were either injected with 0.9% normal saline (control mice) or infected with RRV on day 3 of life and treated subcutaneously with isotype antibodies daily or with 10 μg of anti-C5 (BB5.1) antibodies until day 19 of life. Livers harvested at day 19 and stained with H&E showed normal liver architecture in saline control mice (A), while livers from RRV-infected mice showed prominent development of portal inflammation (B). Daily treatments of BB5.1 significantly attenuated portal inflammation (C). Mag.: 100×.

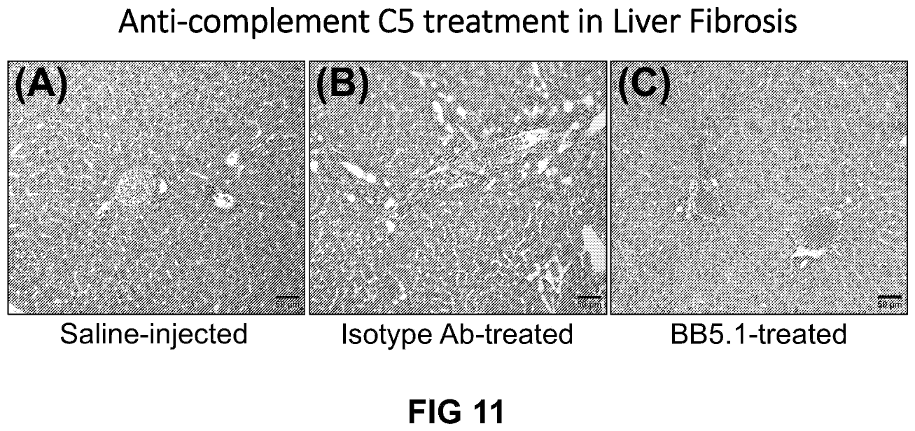

FIG. 12. Anti-complement C5 treatment blocks development of infantile liver fibrosis. Neonatal mice were either injected with 0.9% normal saline (control mice) or infected with RRV on day 3 of life and treated subcutaneously with isotype antibodies daily or with 10 μg of anti-C5 (BB5.1) antibodies until day 19 of life. Livers harvested at day 19 and stained with Sirius Red showed normal liver architecture in saline control mice (A), while livers from RRV-infected mice showed prominent development of portal biliary fibrosis (B). However, daily treatment with BB5.1 antibodies significantly attenuated Sirius Red-positive areas of biliary fibrosis and normalized liver architecture (C). Mag.: 100×.

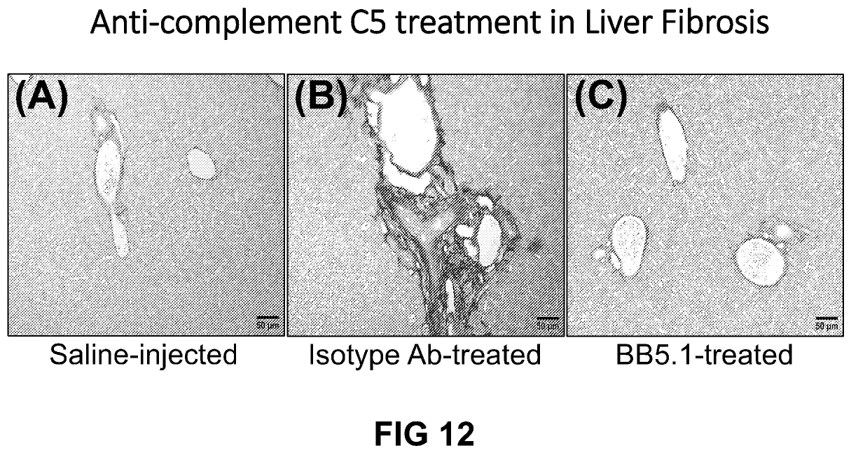

FIG. 13. Blocking Complement C5a promotes survival in mice with liver fibrosis. Neonatal mice were infected with RRV on day 3 of life and treated either untreated, treated subcutaneously with isotype antibodies or with daily 13.5 g of anti-C5a (CLS26) antibodies until day 19 of life. (A) While CLSO26 treatments did not significantly affect weight gain or growth profiles (squares vs circles), anti-C5a therapy significantly suppressed mortality and promoted long-term survival (†) assessed by Kaplan-Meier analysis; isotype-treated or untreated mice (†) showed ˜29% mortality by day 19 after infection. *P≤0.05: RRV-isotype/untreated vs CLSO26-treated mice.

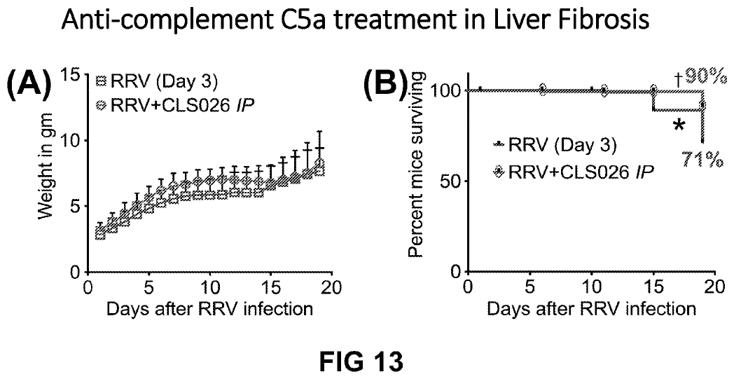

FIG. 14. Anti-complement C5a treatment blocks development of portal inflammation. Neonatal mice were either injected with 0.9% normal saline (control mice) or infected with RRV on day 3 of life and treated subcutaneously with isotype antibodies daily or with 13.5 μg of anti-C5a (CLSO26) antibodies until day 19 of life. Livers harvested at day 19 and stained with H&E showed normal liver architecture in saline control mice (A), while livers from RRV-infected mice showed prominent development of portal inflammation (B). Daily treatments of CLSO26 significantly attenuated portal inflammation (C). Mag.: 100×.

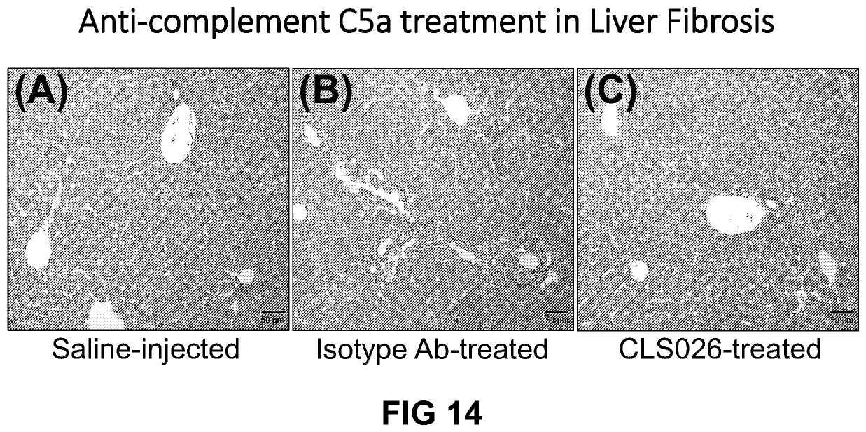

FIG. 15. Anti-complement C5a treatment attenuates infantile liver fibrosis. Neonatal mice were either injected with 0.9% normal saline (control mice) or infected with RRV on day 3 of life and treated subcutaneously with isotype antibodies daily or with 13.5 μg of anti-C5a (cls026) antibodies until day 19 of life. Livers harvested at day 19 and stained with Sirius Red showed normal liver architecture in saline control mice (A), while livers from RRV-infected mice showed prominent development of portal and bridging liver fibrosis (B). However, daily treatment with CLS026 antibodies significantly attenuated Sirius Red-positive areas of biliary fibrosis and normalized liver architecture (C). Mag.: 100×.

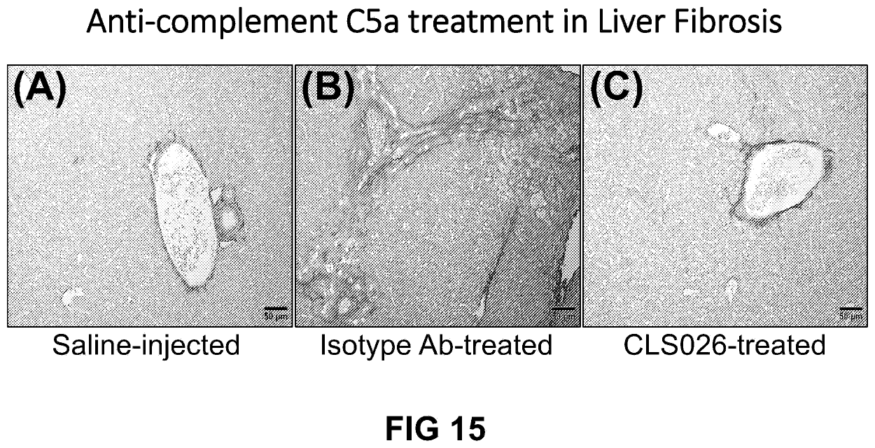

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “preventing” is art-recognized, and when used in relation to a condition, such as a local recurrence, a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to a pediatric individual.

Provided herein are therapeutic compositions and methods for treating or ameliorating biliary atresia, for example, treatment or prevention or amelioration, or attenuation of hepatic fibrosis and/or cirrhosis associated with or caused by biliary atresia. The disclosed methods may comprise administering an effective amount of a C5 inhibitor, more particularly an anti-C5 antibody such as eculizumab or an eculizumab variant, to an individual in need thereof, wherein the patient is one having biliary atresia. In one aspect, the method may comprise treating a subject, in particular a pediatric human subject, having biliary atresia, or having undergone a procedure for biliary atresia (Kasai Procedures), comprising the step of administering a therapeutically effective amount of a C5 inhibitor to the subject in need thereof.

Anti-C5 antibodies are known in the art, and described, for example, in U.S. Pat. Nos. 5,853,722, 6,355,245, 6,355,245, and 10,125,191. The anti-C5 antibody may be a monoclonal antibody, although polyclonal antibodies may also be used. Hybridomas producing monoclonal antibodies reactive with complement component C5 can be obtained according to the teachings of Sims, et al., U.S. Pat. No. 5,135,916. An antibody of the present application can be specific to C5 such that it prevents the cleavage of C5 into C5a and C5b. The antibody can be specific to the C5 convertase. Alternatively, the antibody may be specific to a component of the complement system, for example, C5a, C5b, or C5b-9, and the antibody specific to the component preferably inhibits the component's function, for example, by blocking the component's binding to its respective receptor, or by blocking its function in activating the subsequent signaling or events in the complement cascade.

In one aspect, provided herein is a method for treating a subject, in particular a pediatric human subject, having biliary atresia, or having undergone a procedure for biliary atresia (Kasai Procedures), comprising non-systemically administering to the distal gastrointestinal tract of the individual in need thereof a therapeutically effective amount of a pediatric dosage form of a C5 inhibitor.

In one aspect, the method may include the step of administering a C5 inhibitor in an amount sufficient to attenuate and/or reverse biliary-atresia associated hepatobiliary injury, for example, one or more of intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis, and combinations thereof, in a subject in need thereof, particularly a pediatric subject, more particularly a subject less than three months of age.

In one aspect, the individual being treated may be one having biliary atresia or post-Kasai biliary atresia, and is typically a pediatric subject.

In one aspect, the biliary atresia may be characterized by one or more symptoms selected from jaundice, pruritis, cirrhosis, hypercholemia, neonatal respiratory distress syndrome, lung pneumonia, increased serum concentration of bile acids, increased hepatic concentration of bile acids, increased serum concentration of bilirubin, hepatocellular injury, liver scarring, liver failure, hepatomegaly, xanthomas, malabsorption, splenomegaly, diarrhea, pancreatitis, hepatocellular necrosis, giant cell formation, hepatocellular carcinoma, gastrointestinal bleeding, portal hypertension, hearing loss, fatigue, loss of appetite, anorexia, peculiar smell, dark urine, light stools, steatorrhea, failure to thrive, and/or renal failure.

In one aspect, the pediatric patient may be a new born, a pre-term new born, an infant, a toddler, a preschooler, a school-age child, a pre-pubescent child, post-pubescent child, an adolescent, or a teenager under the age of eighteen. In some embodiments, the pediatric patient is a new born, a pre-term new born, an infant, a toddler, a preschooler, or a school-age child. In some embodiments, the pediatric patient is a new born, a pre-term new born, an infant, a toddler, or a preschooler. In some embodiments, the pediatric patient is a newborn, a pre-term newborn, an infant, or a toddler. In some embodiments, the pediatric patient is a new born, a pre-term new born, or an infant. In some embodiments, the pediatric patient is a new born. In some embodiments, the pediatric patient is an infant. In some embodiments, the pediatric patient is a toddler.

In one aspect, the administration step may attenuate and/or reverse a symptom of biliary atresia or post-Kasai Procedure biliary atresia, for example intrahepatic and/or hepatobiliary inflammation, intrahepatic and/or hepatobiliary fibrosis, cholangiopathy, periductal inflammation, fibrosis, ballooning degeneration, confluent necrosis, portal inflammation, lobular inflammation, bile duct injury, bile duct fibrosis, portal and and/or pericellular bile duct fibrosis, and combinations thereof, in a subject in need of such treatment.

In one aspect, the administration step may promote a regenerative response in a liver and/or a bile duct cell in a subject having biliary atresia. This response may be achieved by administration of a C5 inhibitor at a time point selected from pre-, post-, or during a Kasai Procedure.

In one aspect, the administration step may preserve, restore, or improve liver function in a subject having biliary atresia. This response may be achieved by administration of a C5 inhibitor at a time point selected from pre-, post-, or during a Kasai Procedure.

In one aspect, the administration step may reduce or eliminate the need for a liver transplant in a subject having biliary atresia or post-Kasai Procedure biliary atresia. This response may be achieved by administration of a C5 inhibitor at a time point selected from pre-, post-, or during a Kasai Procedure.

The timing of administering a therapeutic to a subject can vary, for example, depending on the identity of the subject or the cholestatic liver disease or condition to be treated or prevented, or both. For example, the administration may occur before the manifestation of biliary atresia, during the manifestation of biliary atresia, or after the manifestation of biliary atresia. In other aspects, the administration may occur before a Kasai Procedure, during a Kasai Procedure, or after a Kasai Procedure.

Where the administration step occur in conjunction with a Kasai procedure, for example, wherein a first dose is administered during the procedure, immediately following the procedure, within 12 hours of the procedure, within 24 hours of the procedure, or within 48 hours of the procedure, or within 72 hours of the procedure, or within 96 hours of the procedure. A second dose may be administered at a second time point, for example, approximately 1 to about 30 days, or from about 2 to about 20 days, or about from about 3 to about 10 days following the procedure. In other aspects, a third dose may be administered. A third dose may be administered at a time point of about 60 to 90 days following the Kasai procedure, or about 70 to 80 days following the Kasai procedure. In other aspects, the dose may be administered daily, or twice a day, for any number of days following the procedure. In one aspect, the C5 inhibitor may be administered until improvement in a phenotypic outcome occurs, for example, a phenotypic outcome such as attenuation or reversal of inflammation and/or fibrosis progression. In one aspect, the C5 inhibitor may be administered in an amount sufficient to reduce serum biomarkers of liver injury selected from ALT, AST and/or Bilirubin.

In one aspect, the C5 inhibitor may be administered at a dose of from about 1 mg/kg to about 10 mg/kg. In one aspect, the C5 inhibitor may be administered intravenously and/or subcutaneously.

The antibody used in the disclosed methods may be specific to C5 such that it prevents the cleavage of C5 into C5a and C5b. The antibody may be specific to the C5 convertase. Alternatively, the antibody may be specific to a component of the complement system, for example, C5a, C5b, or C5b-9, and the antibody specific to the component preferably inhibits the component's function or by blocking its function in activating the subsequent signaling or events in the complement cascade. Certain embodiments may employ eculizumab or pexelizumab, or both. An antibody or antibody therapeutic of the present application can be a full length immunoglobulin, a monoclonal antibody, a chimeric antibody (e.g., a humanized antibody), a single chain antibody, a domain antibody, an Fab fragment, or an antibody having an Fab fragment and a mutated Fc portion.

In one aspect the antibody used is Eculizumab. Eculizumab is a humanized anti-human C5 monoclonal antibody (Alexion Pharmaceuticals, Inc.), with a human IgG2/IgG4 hybrid constant region, so as to reduce the potential to elicit proinflammatory responses. Eculizumab has the trade name Soliris® and is currently approved for treating paroxysmal nocturnal hemoglobinuria (“PNH”) and atypical hemolytic uremic syndrome (“aHUS”). Eculizumab specifically binds to human C5 protein and blocks the formation of the generation of the potent proinflammatory protein C5a. Eculizumab further blocks the formation of the terminal complement complex.

In some embodiments, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of a active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating a subject having biliary atresia, comprising administering a therapeutically effective amount of a C5 inhibitor to said subject.

2. The method of claim 1, wherein said biliary atresia is post-Kasai biliary atresia.

3. The method of claim 1, wherein said C5 inhibitor is Eculizumab.

4. The method of claim 1, wherein said treatment causes one or both of attenuation and reversal of biliary atresia-associated hepatobiliary injury in said subject.

5. The method of claim 1, wherein said administration promotes a regenerative response in one or both of a liver and a bile duct cell in said subject.

6. The method of claim 1, wherein said administration preserves, restores, or improves liver function in said subject.

7. The method of claim 1, wherein said administration reduces the need for a liver transplant in said subject.

8. The method of claim 1, wherein said administration occurs after said subject has undergone a Kasai procedure.

9. The method of claim 1, wherein said C5 inhibitor is administered in an amount sufficient to reduce a serum biomarker of liver injury selected from one or more of ALT, AST and Bilirubin.

10. The method of claim 1, wherein said C5 inhibitor is administered until improvement in a phenotypic outcome occurs.

11. The method of claim 1, wherein said C5 inhibitor is administered via a route selected from one or both of intravenously and subcutaneously.

12. (canceled)

13. (canceled)

14. (canceled)

15. (canceled)

16. (canceled)

17. (canceled)

18. (canceled)

19. The method of claim 1, wherein the subject is a human subject.

20. The method of claim 1, wherein the subject is a pediatric human subject.

21. The method of claim 4 wherein said biliary-atresia associated hepatobiliary injury is selected from one or more of intrahepatic fibrosis, hepatobiliary fibrosis, cholangiopathy, periductal inflammation, periductal fibrosis, ballooning degeneration, confluent necrosis, portal inflammation, lobular inflammation, bile duct injury, bile duct fibrosis, portal duct fibrosis, and pericellular bile duct fibrosis.

22. The method of claim 8 wherein said administration occurs at the time of said Kasai procedure.

23. The method of claim 8 wherein said administration occurs within about 1 to about 72 hours of said Kasai procedure.

24. The method of claim 8 wherein said administration occurs within about 8 to 36 hours of said Kasai procedure.

25. The method of claim 8 wherein said administration occurs within about 48 hours of said Kasai procedure.

26. The method of claim 10, wherein said phenotypic outcome is selected from one or more of attenuation of inflammation, reversal of inflammation, attenuation of fibrosis progression, and reversal of fibrosis progression.

27. The method of claim 1, wherein treatment with the C5 inhibitor results in improvement in at least one clinical presentation in the subject having biliary atresia, wherein the clinical presentation is selected from (a) extrahepatic bile duct (EHBD) obstruction and atresia; (b) liver fibrosis; (c) portal inflammation; (d) growth failure; and (e) diminished survival.
